SEQUENCE LISTING (30 sequences)

SEQ ID NO: 1 - DNA, 20 bp, Artificial Sequence
  Synthetic
gcaatcctaagttaatggtg

SEQ ID NO: 2 - DNA, 20 bp, Artificial Sequence
  Synthetic
atcaagactgaatgattagc

SEQ ID NO: 3 - DNA, 20 bp, Artificial Sequence
  Synthetic
ctataaagtggtttggccat

SEQ ID NO: 4 - DNA, 18 bp, Artificial Sequence
  Synthetic
gactcgcagtgttgcttg

SEQ ID NO: 5 - DNA, 20 bp, Artificial Sequence
  Synthetic
ctggcttcacaatgaataat

SEQ ID NO: 6 - DNA, 19 bp, Artificial Sequence
  Synthetic
ctaaggtaggtgcacagat

SEQ ID NO: 7 - DNA, 20 bp, Artificial Sequence
  Synthetic
gccgatgaacataaacaaga

SEQ ID NO: 8 - DNA, 20 bp, Artificial Sequence
  Synthetic
tctctgagaatgggcttaga

SEQ ID NO: 9 - DNA, 20 bp, Artificial Sequence
  Synthetic
gaaccatgtggaaagacttg

SEQ ID NO: 10 - DNA, 20 bp, Artificial Sequence
  Synthetic
ctttggttctcataaatgcc

SEQ ID NO: 11 - DNA, 18 bp, Artificial Sequence
  Synthetic
cacatcaccaccatcttc

SEQ ID NO: 12 - DNA, 20 bp, Artificial Sequence
  Synthetic
tagacgtgtcaggagttgtg

SEQ ID NO: 13 - DNA, 20 bp, Artificial Sequence
  Synthetic
gtagatgaagctggaagtgg

SEQ ID NO: 14 - DNA, 18 bp, Artificial Sequence
  Synthetic
ttcagctttgggtgtagg

SEQ ID NO: 15 - DNA, 18 bp, Artificial Sequence
  Synthetic
aactacaactgctgcacc

SEQ ID NO: 16 - DNA, 18 bp, Artificial Sequence
  Synthetic
ggttttctcactctaggc

SEQ ID NO: 17 - DNA, 20 bp, Artificial Sequence
  Synthetic
aaaagccaaccaaagcaccc

SEQ ID NO: 18 - DNA, 20 bp, Artificial Sequence
  Synthetic
gtagtcagtccatctactgg

SEQ ID NO: 19 - DNA, 18 bp, Artificial Sequence
  Synthetic
ttggaggactaactggac

SEQ ID NO: 20 - DNA, 18 bp, Artificial Sequence
  Synthetic
ctttggataaggtctgcc

SEQ ID NO: 21 - DNA, 20 bp, Artificial Sequence
  Synthetic
cagaaaacctgacggctatg

SEQ ID NO: 22 - DNA, 20 bp, Artificial Sequence
  Synthetic
tttacaggtgtgagccatgc

SEQ ID NO: 23 - DNA, 23 bp, Homo sapiens
ctgcacctaagatgacaaaagag

SEQ ID NO: 24 - DNA, 18 bp, Homo sapiens
ctgcacctgacaaaagag

SEQ ID NO: 25 - DNA, 28 bp, Homo sapiens
tccaaactccaaactagttgaagtaaat

SEQ ID NO: 26 - DNA, 21 bp, Homo sapiens
tccaaactagttgaagtaaat

SEQ ID NO: 27 - protein, 792 aa, Homo sapiens
  NON_CONS (267)..(268)
  NON_CONS (321)..(322)
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNKEPAPTTTKEPAPTTPKEPAPTTTKEPAPTTTKSAPTTPKEPAPTTPKKPAPTTAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGEMTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 28 - protein, 71 aa, Bos taurus
GRCGEGYSRDAICNCDYNCQHYMECCPDFKKECTVELSCKGRCFETFARGRECDCDSDCKKYGKCCPDYES

SEQ ID NO: 29 - protein, 401 aa, Bos taurus
EFPVPKGRATNSQVTTPKPQKPTKAPKKPTSTKKPRTPRVRKPKTTPTPPKTTTSAMPEPTPTSLPEAMLQTTTRPTPTPNSEIIDVNSENEDGDAAEGEKPHMIFRPPVLTPIVIPGTEIIVRGPSQGFGINPMFSDETNLCNGRPVDGLTTLRNGTLVAFRGHYFWMLTPFTPPPPPRRITEVWGIPSPIDTVFTRCNCEGKTFFFKGSQYWRFTNDIKDAGYPKLISKGFGGLNGKIVAALSIAQYKSRPESVYFFKRGGSVQQYTYKQEPTQKCTGRRPAINYSVYGETAQVRRRRFERAIGPSQVHTIRIHYTPVRVPYQDKGFLHNEVKVSTLWRGLPNVVTSAISLPNIRKPDGYDYYALSKDQYYNIDVPSRTARAITTRSGQTLSNTWYNCP

SEQ ID NO: 30 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTAPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGEMTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

(19) United States
(12) Patent Application Publication
(10) Pub. No.: US 2002/0086824 A1
(43) Pub. Date: Jul. 4, 2002

(54) Novel methods and reagents for the treatment of osteoarthritis

(75) Inventors: Matthew L. Warman, Cleveland, OH (US); John D. Carpten, Gaithersburg, MD (US); Jeffery M. Trent, Rockville, MD (US); Jose Marcelino, South Euclid, OH (US)

Correspondence Address: Peter G. Carroll, MEDLEN & CARROLL, LLP, Suite 350, 101 Howard Street, San Francisco, CA, 94104, US

(73) Assignee: Case Western Reserve University, Cleveland, OH (US)

(21) Appl. No.: 09/802,207

(22) Filed: Mar. 8, 2001

Related U.S. Application Data
(63) Continuation of application No. 09/619,175, filed on Jul. 19, 2000.
(60) Provisional application No. 60/145,328, filed on Jul. 23, 1999.

(51) Int. Cl. A61K 38/17

(52) U.S. Cl. 514/12

(57) ABSTRACT

Methods and compositions are described for treating osteoarthritis. Treatment is described with a new class of anti-OA drug, namely compounds that may be used as lubricants of the tissue diagnosed with OA. Additionally, the present invention provides reagents for the screening of compounds that may be used as therapeutic agents in the treatment of OA.

This invention was made in part with government support under grant AR43827 from the National Institutes of Health. The government has certain rights in the invention.

### FIELD OF THE INVENTION

This invention generally relates to novel compounds that may be used as lubricants of tissue and joints. Additionally, the present invention provides reagents for the screening of compounds that may be used as therapeutic agents in the treatment of osteoarthritis.

### BACKGROUND

Osteoarthritis (OA) is a degenerative disorder of joints and cartilage. The articular surfaces are disrupted involving a loss of normal collagen architecture and a chondrocyte response that replaces the abnormal structure. The replacement cartilage is less resistant to wear than the original and the progression of OA eventually results in a complete loss of any articular joint protection by the extracellular matrix. (LaPrade, R. F. and Swiontkowski, M. F., “New horizons in the treatment of osteoarthritis of the knee” JAMA 281:876-878, 1999)

The hereditary nature of OA was first reported in the 1940's. The most genetically susceptible constituents of cartilage function in OA include; 1) the functional organization of macromolecular elements of the cartilage determined by specific associations between proteins, proteoglycans, and cells, 2) alterations of collagen and proteoglycan side-chains which are responsible for the structural integrity of the joint, and 3) proteins involved in intracellular signalling processes which affect chondrocyte synthesis and catabolism of matrix components. Genetic models linked to OA have focused on the fibrillar collagens of types II, V, and XI. Of these reports, mutations in type II collagen are most common. These single point mutations usually involve a C to T substitution and effect an obligatory glycine. The triple helix of collagen requires close packing, and the substitution of glycine for a large, sterically bulky or highly charged, side chain amino acid disrupts the necessary quaternary structure assembly. Similarly, current investigations using transgenic knockout mice focus on the cartilage matrix proteins. (Holderbaum, D. and Haqqi, T. M. et al., “Genetics and osteoarthritis: exposing the iceberg” Arthritis Rheum 42:397-405, 1999)

The primary clinical symptom of OA is joint pain related to physical activity. In active and progressive OA, treatment of these symptoms with non-steroidal anti-inflammatory drugs (NSAIDs) ultimately fails resulting in a requirement for complete joint replacement. The available palliative effects from NSAIDs do not provide adequate pain relief or amelioration of other symptoms thus stimulating the development of alternative treatments.

The administration of hyaluronic acid (HA) has spread from Europe and Canada to the United States and has received FDA approval for use in advanced OA conditions. The principle of HA administration is to restore the normal viscoelastic properties of synovial fluid that relieves the signs and symptoms of OA. (LaPrade, R. F. and Swiontkowski, M. F., “New horizons in the treatment of osteoarthritis of the knee” JAMA 281:876-878, 1999) A critical review of studies that HA achieves significant analgesic and anti-inflammatory relief in OA patients questions the appropriateness of HA for long-term successful therapy. (Simon, L. S., “Visco-supplementation therapy with intra-articular hyaluronic acid. Fact or fantasy?” Rheum Dis Clin North Am25:345-357, 1999)

For example, the administration of HA to four patients with sacroiliac involvement was effective in relieving pain only half the time. (Srejic, U. and Calvillo, O., et.al., “Visco-supplementation: a new concept in the treatment of sacroiliac joint syndrome; a preliminary report of four cases” Reg Anesth Pain Med 24:84-88, 1999) Treatment of OA with HA viscosupplementation is most useful when other medical forms of therapy are contraindicated, toxic, or have failed. HA treatment is not expected to replace the need for thigh muscle strengthening or for overweight patients to lose weight. Whether or not efficacy will demand or warrant earlier or repeated use of HA-like products is not clear. (Cohen, M. D., “Hyaluronic acid treatment (viscosupplementation) of OA of the knee” Bull Rheum Dis 47:4-7, 1998) The intracellular mechanism of HA is not well known. HA is able to modulate a variety of cellular functions, suppress the activities of pro-inflammatory mediators, or attenuate nociceptive responses. However, recent studies with animal models of non-inflammatory OA have questioned the ability of HA to protect articular cartilage degeneration directly. (Ghosh, P., “The role of hyaluronic acid (hyaluronan) in health and disease: interactions with cells, cartilage, and components of synovial fluid” Clin Exp Rheumatol 12:75-82, 1994) Interestingly, HA is implicated in the efficacy of glucosamine administration to OA patients. The traditional explanation of glucosamine therapy is that it promotes the synthesis of cartilage proteoglycans. However, the rapid symptomatic response to high-dose glucosamine in OA patients is not consistent with this mechanism. An alternative or additional possibility is that glucosamine stimulates synovial production of HA. (McCarty, M. F., “Enhanced synovial production of hyaluronic acid may explain rapid clinical response to high-dose glucosamine in osteoarthritis” Med Hypotheses 50:507-510, 1998; Kelly, G. S., “The role of glucosamine sulfate and chondroitin sulfates in the treatment of degenerative joint disease” Altern Med Rev 3:27-39, 1998) Regardless of its mechanism, researchers have expressed the view that the proper studies are lacking that can place glucosamine in it's appropriate place in the therapeutic armamentarium of OA. (da Camara, C. C. and Dowless, G. V., “Glucosamine sulfate for osteoarthritis” Ann Pharmacother 32:580-587, 1998)

Specific clinical trials using galatosaminoglycuronglycan exemplifies the above overall doubts concerning the curative efficacy of glucosamines. In patients with erosive OA the administration of galatosaminoglycuronglycan only provided pain relief and did not significantly improve the clinical aspects of reduced joint space and erosive progression. (Rovetta, G. and Monteforte, P., “Galatosaminoglycuronglycan sulfate in erosive osteoarthritis of the hands:early diagnosis, early treatment” Int J Tissue React 18:43-46, 1996) Another, long-term, clinical trial provides data showing that after three years of a glycosaminoglycan-peptide treatment, 84% of the patients had either no change or actual deterioration when comparing pre- and post-treatment joint radiology examinations. (Katona, K., “A clinical trial of glycosaminoglycan-peptide complex (‘Rumalon’) in patients with osteoarthritis of the knee” Curr Med Res Opin 10:625-633, 1987) Similarly, an eight week trial comparing glucosamine to ibuprofen indicated an enhanced response of glucosamine only for pain relief and not swelling or any other measured parameter. (Lopes-Vaz, A., “Double-blind clinical evaluation of the relative efficacy of ibuprofen and glucosamine sulphate in the management of osteoarthritis of the knee in out-patients” Curr Med Res Opin 8:145-149, 1982)

Thus, there is a need for better treatment approaches to OA. Ideally, such approaches should alleviate the clinical causality, instead of only the symptomology, without causing any undue side effects.

### SUMMARY OF THE INVENTION

This invention generally relates to novel compounds that may be used as lubricants of tissue and joints. Additionally, the present invention provides reagents for the screening of compounds that may be used as therapeutic agents in the treatment of Osteoarthritis. In one embodiment, the present invention contemplates the CACP protein, or portions thereof, in a preparation suitable for use as a lubricant. The present invention contemplates that such a preparation can be used in a method of treatment. In one embodiment, the method comprises a) providing: i) a subject (e.g. a human or animal), and ii) a preparation comprising the CACP protein, or portion thereof, and b) administering said preparation to said subject to lubricate the subjects tissue or joints. In another embodiment, the method comprises a) providing: i) a subject (e.g. a human or animal) diagnosed with arthritis, and ii) a preparation comprising the CACP protein, or portion thereof, and b) administering said preparation to said subject. In yet another embodiment, the method comprises a) providing: i) a subject (e.g. a human or animal) with symptoms of osteoarthritis, and ii) a preparation comprising the CACP protein, or portion thereof; and b) administering said preparation to said subject under conditions such that said symptoms (e.g. joint pain, loss of range of movement, joint damage, etc.) are reduced. In all of the above methods, it is contemplated that the preparation can have other ingredients. In one embodiment, said preparation further comprises a local anesthetic. Thus, the present invention contemplates a composition, comprising CACP protein, or portion thereof, in combination with an anesthetic.

It is not intended that the present invention be limited to the particular mode of administering the above-noted preparation. In one embodiment, said administering comprises intra-articular injection. In another embodiment, said administering comprises intravenous injection. In yet another embodiment, said preparation is administered topically. Such topically administered preparations may have ingredients that permit penetration of the skin (e.g. DMSO).

### DESCRIPTION OF THE FIGURES

FIG. 1 shows the clinical features of camptodactyly-arthropathy-coxa vara-pericarditis syndrome (“CACP”). Part a) is from a synovial biopsy (200 × magnification) showing hyperplasia of synoviocytes (between arrowheads) without evidence of inflammation. The joint cavity is on the right. In contrast to the normal synoviocyte layer which is 1-3 cells deep, the layer here is 3-10 cells deep. Part b) illustrates hands showing flexion deformity of the proximal interphalangeal joints of all fingers as well as the distal interphalangeal joint of the thumb finger (arrow). Also note the bilateral swelling at the wrists (arrows). c) lower extremities showing swelling of the knees and ankles. d) pericardial biopsy (10 × magnification) showing hyperplasia of the intimal cells (between arrowheads). The pericardial cavity is on the right. The subintimal fibrous layer is also thickened.

FIG. 2 shows a schematic of the CACP proteoglycan and the putative effects of each mutation. Part a) is full length protein showing regions of homology to other protein families. Lettered and numbered bars below the schematic indicate the PCR amplimers evaluated for mutations from patient-derived cDNA and genomic DNA, respectively (i.e. lettered bars correspond to amplimers derived from cDNA and numbered bars correspond to individual CACP exons that were amplified and sequenced from genomic DNA). b) schematic depicting the predicted protein product in patients with the 5 bp deletion. c) segregation of the 5 bp deletion with the phenotype. Unaffected parents are heterozygous for mutant and wild type alleles while the affected patients are homozygous for the mutant allele. d) chromotograms of wild type and mutant alleles. Boxed area indicates the nucleotide residues deleted in the affected patients. e) schematic depicting the predicted protein product in patients with the 7 bp deletion. f) chromatograms of wild type and mutant alleles. Boxed area indicates the nucleotide residues deleted in the affected patients. g) schematic depicting the predicted protein product in the patient with the 41 bp splice site insertion. h) schematic depicting the predicted protein product in the patient heterozygous for a C to T transition creating a stop codon.

FIG. 3 shows northern blots of CACP in synovial tissue and in other tissues. a) Bovine tissue northern blot demonstrating strong expression of CACP mRNA in synovial tissue, and weaker expression in pericardial tissue and isolated chondrocytes from articular cartilage. 5 μg of total RNA is loaded onto each lane. b) Multi-tissue northern blot demonstrating CACP expression. Above) the 4.5 kb CACP mRNA transcript is expressed in liver (signal is easily detected after a 24 hour exposure using X-ray film). Below) control hybridization using an actin probe demonstrating approximately uniform mRNA loading in all non-muscle containing lanes. 2 μg of poly-A+ RNA is loaded onto each lane.

FIG. 4 shows a sequence alignment between MSF and SZP

### DEFINITIONS

To facilitate understanding of the invention, a number of terms are defined below.

The term “homology” when used in relation to proteins refers to a degree of complementarity. There may be partial homology or complete homology (i.e., identity). A partially complementary sequence is one that at least partially inhibits a completely complementary sequence from performing its function (e.g. enzymatic, binding, etc) in vivo or in vitro and is referred to using the functional term “substantially homologous.” The inhibition function of the completely complementary sequence may be examined using an enzymatic assay, a binding assay or other assay designed to measure the particular function of the completely complementary protein.

The present invention contemplates CACP nucleic acid amplified from genomic DNA and mRNA, and substantially homologous sequences. A “substantially homologous sequence” or probe will compete for and inhibit the function (e.g., the binding or enzymatic function) of a sequence which is completely homologous to a target under conditions of low stringency. This is not to say that conditions of low stringency are such that non-specific interaction is permitted; low stringency conditions require that the interaction of the sequence with its substrate be a specific (i.e., selective) interaction. The absence of non-specific binding may be tested by the use of a second target which lacks even a partial degree of complementarity (e.g., less than about 30% identity); in the absence of non-specific interaction the probe will not react to the second non-complementary target.

Low stringency conditions when used in reference to nucleic acid hybridization comprise conditions equivalent to binding or hybridization at 42° C. in a solution consisting of 5× SSPE (43.8 g/l NaCl, 6.9 g/l NaH₂PO₄·H₂O and 1.85 g/l EDTA, pH adjusted to 7.4 with NaOH), 0.1% SDS, 5X Denhardt's reagent [50X Denhardt's contains per 500 ml: 5 g Ficoll (Type 400, Pharmacia), 5 g BSA (Fraction V; Sigma)] and 100 μg/ml denatured salmon sperm DNA followed by washing in a solution comprising 5X SSPE, 0.1% SDS at 42° C. when a probe of about 500 nucleotides in length is employed.

High stringency conditions when used in reference to nucleic acid hybridization comprise conditions equivalent to binding or hybridization at 42° C. in a solution consisting of 5X SSPE (43.8 g/l NaCl, 6.9 g/l NaH₂PO₄·H₂O and 1.85 g/l EDTA, pH adjusted to 7.4 with NaOH), 0.5% SDS, 5X Denhardt's reagent and 100 μg/ml denatured salmon sperm DNA followed by washing in a solution comprising 0.1X SSPE, 1.0% SDS at 42° C. when a probe of about 500 nucleotides in length is employed.

When used in reference to nucleic acid hybridization the art knows well that numerous equivalent conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high stringency hybridization different from, but equivalent to, the above listed conditions.

“Stringency” when used in reference to nucleic acid hybridization typically occurs in a range from about Tₘ−5° C. (5° C. below the Tₘ of the probe) to about 20° C. to 25° C. below Tₘ. As will be understood by those of skill in the art, a stringent hybridization can be used to identify or detect identical polynucleotide sequences or to identify or detect similar or related polynucleotide sequences. Under “stringent conditions” a nucleic acid sequence of interest will hybridize to its exact complement and closely related sequences.

As used herein, the term “fusion protein” refers to a chimeric protein containing the protein of interest (i.e., CACP and fragments thereof) joined to an exogenous protein fragment (the fusion partner which consists of a non-CACP sequence). The fusion partner may provide a detectable moiety, may provide an affinity tag to allow purification of the recombinant fusion protein from the host cell, or both. If desired, the fusion protein may be removed from the protein of interest by a variety of enzymatic or chemical means known to the art.

As used herein, the term “purified” or “to purify” refers to the removal of contaminants from a sample. The present invention contemplates purified compositions (discussed above).

As used herein, the term “partially purified” refers to the removal of a moderate portion of the contaminants of a sample to the extent that the substance of interest is recognizable by techniques known to those skilled in the art as accounting for a measurable amount of the mixture.

As used herein, the term “substantially purified” refers to the removal of a significant portion of the contaminants of a sample to the extent that the substance of interest is recognizable by techniques known to those skilled in the art as the most abundant substance in the mixture. The present invention contemplates purified , partially purified, and substantially purified CACP gene product, and portions thereof for use as a lubricant.

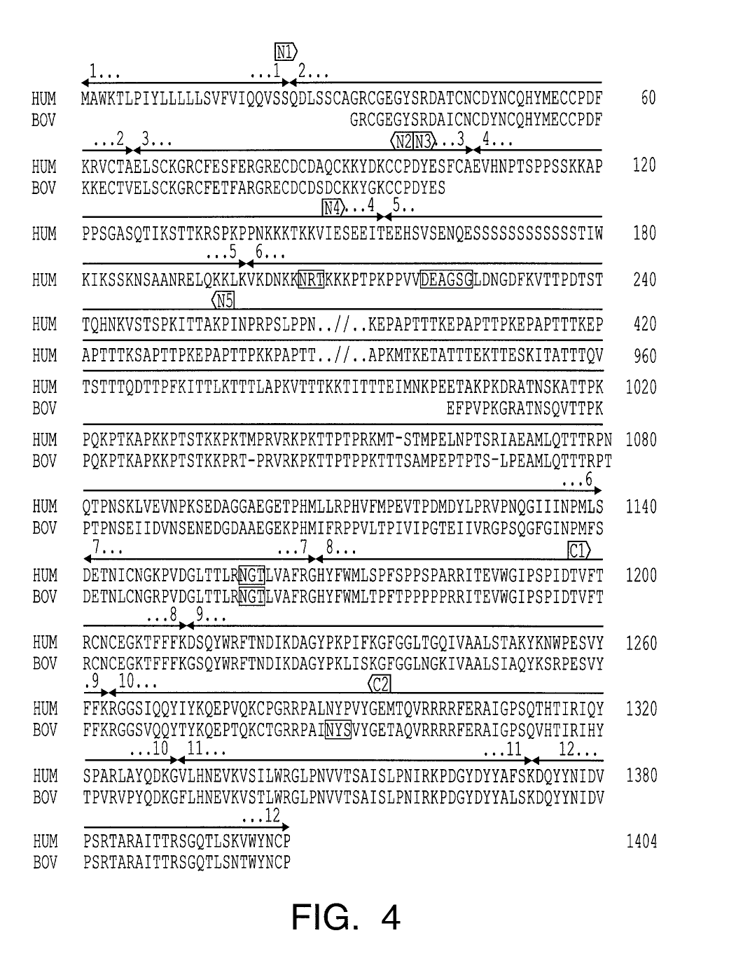

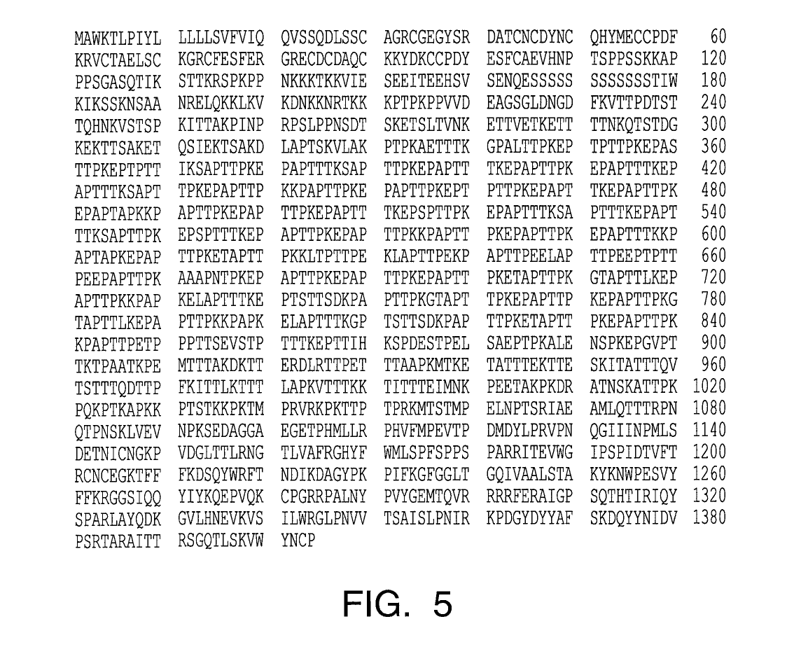

As used herein the term “portion” when in reference to a protein (as in “a portion of a given protein”) refers to fragments of that protein. The fragments may range in size from four amino acid residues to the entire amino acid sequence minus one amino acid. In one embodiment, the present invention contemplates “functional portions” of a protein. Such portions are “functional” if they contain a binding region (i.e. a region having affinity for another molecule) and such binding can take place (i.e. the binding region functions, albeit with perhaps lower affinity than that observed for the full-length protein). Such “functional portions” of the CACP gene product are typically greater than 50 amino acids in length, and more typically greater than 100 amino acids in length. “Functional portions” may also be “conserved portions” of the protein. The present invention contemplates conserved portions 20 amino acids in length or greater. The alignment shown in FIG. 4 permits the selection of particular embodiments of conserved portions.

As used herein the term “portion” when in reference to an oligonucleotide sequence (as in “a portion of a given sequence”) refers to fragments of that sequence. The fragments may range in size from four base residues to the entire oligonucleotide sequence minus one base. More typically, such portions are 15 nucleotides in length or greater. Again, such portions may be conserved portions. On the other hand, such portions may be unique portions of the gene.

“Staining” shall be defined as any number of processes known to those in the field that are used to better visualize, distinguish or identify a specific component(s) and/or feature(s) of a cell or cells.

“Morphology” shall be defined as the visual appearance of a cell or organism when viewed with the eye, a light microscope, a confocal microscope or an electronmicroscope, as appropriate.

“In operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. For example, the present invention contemplates the CACP gene in operable combination with a promoter. The term also refers to the linkage of amino acid sequences in such a manner so that a functional protein is produced.

“Heterologous DNA” sequence refers to a nucleotide sequence which is not endogenous to the cell into which it is introduced. Heterologous DNA includes a nucleotide sequence which is ligated to, or is manipulated to become ligated to, a nucleic acid sequence to which it is not ligated in nature, or to which it is ligated at a different location in nature. Heterologous DNA also includes a nucleotide sequence which is naturally found in the cell into which it is introduced and which contains some modification relative to the naturally-occurring sequence. An example of heterologous DNA of the present invention comprises the CACP DNA sequence introduced into yeast.

“Expression vector” shall be defined as a sequence of DNA or RNA, in operable combination that is used to transfect a cell or cells. The sequence may be single or double stranded. For example, the present invention contemplates an expression vector comprising the CACP gene.

“Patient” shall be defined as a human or other animal, such as a guinea pig or mouse and the like, capable of having cell cycle (influenced) determined diseases, either naturally occurring or induced, including but not limited to cancer.

### GENERAL DESCRIPTION OF THE INVENTION AND PREFERRED EMBODIMENTS

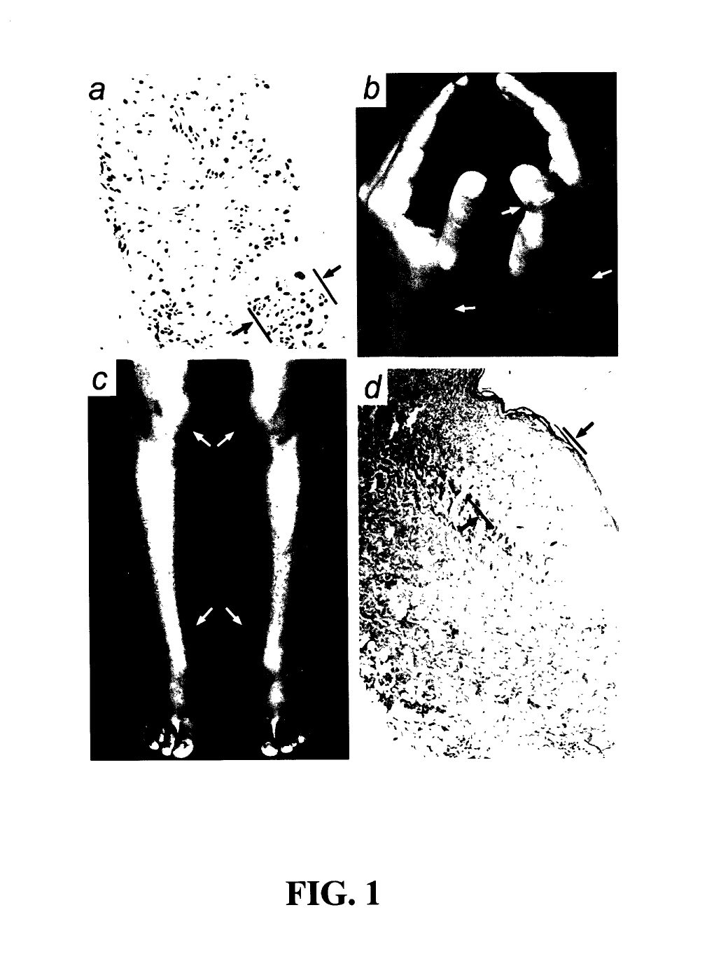

Synovium is a specialized tissue that nourishes and lubricates joints and tendons. Synovium also clears metabolites that accumulate in Joint cavities (Levick, J. R. “Blood flow and mass transport in synovial joints” In Handbook of Physiology Vol. IV, Microcirculation, Part 2. Edited by E. M. Renkins, C. C. Michel, Bethesda, Md., Am. Physiological Society pp 917-947, 1984). Hyperplasia of synoviocytes in the context of inflammation is a characteristic feature of rheumatoid arthritis (Harris, E. D. “Mechanisms of disease: Rheumatoid arthritis-pathophysiology and implications for therapy” New Engl. J. Med. 322:1277-1289, 1990), in which synoviocyte overgrowth may contribute to joint destruction by interfering with the normal exchange of nutrients and waste products between the vascular/lymphatic plexus and the joint cavity (Wallis, W. J., et al. “Low synovial clearance of iodide provides evidence of hypoperfusion in chronic rheumatoid synovitis” Arthritis Rheum 28:1096-1104, 1985). Hyperplastic synoviocytes may also directly damage articular cartilage by producing degradative enzymes (Case, J. P., et al. “Transin/stromelysin expression in rheumatoid synovium. A transformation-associated metalloproteinase secreted by phenotypically invasive synoviocytes” Am. J. Pathol 135:1055-1064, 1989) and by invading the articular cartilage surface (Firestein, G. S. “Invasive fibroblast-like synoviocytes in rheumatoid arthritis. Passive responders or transformed aggressors?” Arthritis Rheum. 39:1781-90, 1996). Patients with the heritable disorder CACP have synovial hyperplasia without evidence of inflammation (FIG. 1a) (Athreya, B. H. and Schumacher, H. R. “Pathologic features of a familial arthropathy associated with congenital flexion contractures of the fingers” Arthritis Rheum. 21:429-437, 1978; Ochi, T., et al. “The pathology of the involved tendons in patients with familial arthropathy and congenital camptodactyly” Arthritis Rheum. 26:896-900, 1983). This results in congenital or childhood-onset camptodactyly (flexion contractures of the interphalangeal joints of fingers and toes) (FIG. 1b) and childhood-onset arthropathy (pain, swelling, and/or restricted range of motion in the large joints) (FIG. 1b,c). Thickening of the pericardium can also occur in CACP (Martinez-Lavin, M. et al. “A familial syndrome of pericarditis, arthritis, and camptodactyly” New Engl. J. Med. 309:224-225, 1983) and is associated with overgrowth of the intimal portion of the fibrous pericardium, again without evidence of inflammation (FIG. 1d). Fibrosing pleuritis has also been reported (Verma, U. N. et al. “A syndrome of fibrosing pleuritis, pericarditis, and synovitis with infantile contractures of fingers and toes in 2 sisters: “familial fibrosing serositis” ” J. Rheumatol. 22:2349-2355, 1995). Pericarditis and pleuritis, in the context of inflammation, occur in patients with rheumatoid arthritis (McRorie, E. R., et al.“Rheumatoid constrictive pericarditis” Br. J. Rheumatol. 36:100-103, 1997; Graham, W. R. “Rheumatoid pleuritis” Southern Med. J. 83:973-975 1990) suggesting that the protein product responsible for causing CACP may also contribute to the pathogenesis of rheumatoid arthritis.

1. Preparation of Synovium Lubricant Compositions

The present invention contemplates preparations comprising synovium lubricants (e.g.

CACP protein, or portions thereof). Said CACP protein may be purified from source tissue (e.g. bovine sources) or produced using recombinant technology (see, generally, Sambrook et Molecular Cloning: A Laboratory Manual, 2d ed. (1989) Cold Spring Harbor Laboratory Press, Cold Spring Harbor, N.Y., and Current Protocols in Molecular Biology (1996) John Wiley and Sons, Inc., N.Y., which are incorporated herein by reference) which is taught provided throughout this document. All the information contained therein is incorporated herein by reference. Formulations can be prepared either as liquid solutions or suspensions, or in solid forms. Formulations may include such normally employed additives such as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained release formulations, or powders, and typically contain 1%-95% of active ingredient, preferably 2%-70%.

The compositions are also prepared as injectables, either as liquid solutions or suspensions; solid forms suitable for solution in, or suspension in, liquid prior to injection may also be prepared. For intra-articular injections (see below), the present invention contemplates formulations comprising one or more synovium lubricants along with one or more local anesthetic. It is not intended that the present invention be limited to particular anesthetics. A variety are contemplated including but not limited to procaine or lidocaine. When injecting a bursa, tendon sheath, or periarticular region, such a mixture will give immediate relief (due to the anesthetic) followed by more lasting relief (due to the lubricant).

Where mixtures with local anesthetics are not desired, a topical anesthetic prior to injection may be used. Such topical anesthetics include but are not limited to ethyl chloride spray on the skin over the joint to be injected. Alternatively, a local anesthetic may be given first, followed by administration of one or more of the above-described lubricants.

2. Delivery Of Formulations And Intra-Articular Injections

It is not intended that the present invention be limited to the particular route of administration. The synovium lubricants can be given orally, applied as creams or ointments or injected (including but not limited to intravenous injection and intra-articular injection). The present invention specifically contemplates intra-articular injections in patients.

To perform an arthrocentesis, the specific area of the joint to be injected is palpated and is then marked, e.g., with firm pressure by a ballpoint pen that has the inked portion retracted. This will leave an impression that will last 10 to 30 minutes. (The ballpoint pen technique can also be used with soft tissue injection.) The area to be aspirated and/or injected should be carefully cleansed with a good antiseptic, such as one of the iodinated compounds. Then the needle can be inserted through the ballpoint pen impression.

Helpful equipment includes the following items: alcohol sponges; iodinated solution and surgical soap; gauze dressings (2×2); sterile disposable 3-, 10- and 20-ml syringes; 18- and 20-gauge, 1 ½-inch needles; 20-gauge spinal needles; 25-gauge, ⅝-inch needles; plain test tubes; heparinized tubes; clean microscope slides and coverslips; heparin to add to heparinized tubes if a large amount of inflammatory fluid is to be placed in the tube; fingernail polish to seal wet preparation; chocolate agar plates or Thayer-Martin medium; tryptic soy broth for most bacteria; anaerobic transport medium (replace periodically to keep culture media from becoming outdated); tubes with fluoride for glucose; plastic adhesive bandages; ethyl chloride; hemostat; tourniquet for drawing of simultaneous blood samples; and 1 percent lidocaine.

Knee. The knee is the easiest joint to inject. The patient should be in a supine position with the knee fully extended. The puncture mark is made just posterior to the medial portion of the patella, and an 18- to 20-gauge, 1 ½-inch needle directed slightly posteriorly and slightly inferiorly. The joint space should be entered readily. On occasion thickened synovium or villous projections may occlude the opening of the needle, and it may be necessary to rotate the needle to facilitate aspiration of the knee when using the medial approach. An infrapatellar plica, a vestigal structure that is also called the ligamentum mucosum, may prevent adequate aspiration of the knee when the medial approach is used. However, the plica should not adversely affect injections or aspirations from the lateral aspect.

Shoulder. Injections in the shoulder are most easily accomplished with the patient sitting and the shoulder externally rotated. A mark is made just medial to the head of the humerus and slightly inferiorly and laterally to the coracoid process. A 20- to 22-gauge, 1 ½-inch needle is directed posteriorly and slightly superiorly and laterally. One should be able to feel the needle enter the joint space. If bone is hit, the operator should pull back and redirect the needle at a slightly different angle.

The acromioclavicular joint may be palpated as a groove at the lateral end of the clavicle just medial to the shoulder. A mark is made, and a 22- to 25-gauge, ⅝- to 1-inch needle is carefully directed inferiorly. Rarely is synovial fluid obtained.

The sternoclavicular joint is most easily entered from a point directly anterior to the joint. Caution is necessary to avoid a pneumotharax. The space is fibrocartilaginous, and rarely can fluid be aspirated.

Ankle Joint. For injections of the lubricants of the present invention in the ankle joints, the patient should be supine and the leg-foot angle at 90 degrees. A mark is made just medical to the tibialis anterior tendon and lateral to the medial malleolus. A 20- to 22-gauge, 1 ½-inch needle is directed posteriorly and should enter the joint space easily without striking bone.

Subtalar Ankle Joint. Again, the patient is supine and the leg-foot angle at 90 degrees. A mark is made just inferior to the tip of the lateral mallcolus. A 20- to 22-gauge, 1 ½-inch needle is directed perpendicular to the mark. With this joint the needle may not enter the first time, and another attempt or two may be necessary. Because of this and the associated pain, local anesthesia may be helpful.

Wrist. This is a complex joint, but fortunately most of the intercarpal spaces communicate. A mark is made just distal to the radius and just ulnar to the so-called anatomic snuff box. Usually a 24- to 26-gauge, ⅝ to 1-inch needle is adequate, and the injection is made perpendicular to the mark. If bone is hit, the needle should be pulled back and slightly redirected toward the thumb.

First Carpometacarpal Joint. Degenerative arthritis often involves this joint. Frequently the joint space is quite narrowed, and injections may be difficult and painful. A few simple maneuvers may make the injection fairly easy, however. The thumb is flexed across the palm toward the tip of the fifth finger. A mark is made at the base of the first metacarpal bone away from the border of the snuff box. A 22- to 26-gauge, ⅝ to 1-inch needle is inserted at the mark and directed toward the proximal end of the fourth metacarpal. This approach avoids hitting the radial artery.

Metacarpophalalangeal Joints and Finger Interphalangral Joints. Synovitis in these joints usually causes the synovium to bulge dorsally, and a 24- to 26-gauge, ½ to ⅝-inch needle can be inserted on the either side just under the extensor tendon mechanism. It is not necessary for the needle to be interposed between the articular surfaces. Some prefer having the fingers slightly flexed when injecting the metacarpophalangeal joints. It is unusual to obtain synovial fluid. When injecting, a mix of the lubricants of the present invention with a small amount of local anesthetic is preferred.

Metatarsophalangeal Joints and Toe Interphalangeal Joints. The techniques are quite similar to those of the metacapophalangeal and finger interphalangeal joints, but many prefer to inject more dorsally and laterally to the extensor tendons. Marking the area(s) to be injected is helpful as is gentle traction on the toe of each joint that is injected.

Elbow. A technique preferred by many is to have the elbow flexed at 90 degrees.

The joint capsule will bulge if there is inflammation. A mark is made just below the lateral epicondyle of the humerus. A 22-gauge, 1 to 1 ½-inch is inserted at the mark and directed parallel to the shaft of the radius or directed perpendicular to the skin.

Hip. This is a very difficult joint to inject even when using a fluoroscope as a guide.

Rarely is the physician quite sure that the Joint has been entered; synovial fluid is rarely obtained. Two approaches can be used, anterior or lateral. A 20-gauge, 3 ½-inch spinal needle should be used for both approaches.

For the anterior approach, the patient is supine and the extremity-fully extended and externally rotated. A mark should be made about 2 to 3 cm below the anterior superior iliac spine and 2 to 3 cm lateral to the femoral pulse. The needle is inserted at a 60 degree angle to the skin and directed posteriorly and medially until bone is hit. The needle is withdrawn slightly, and possibly a drop or two of synovial fluid can be obtained, indicating entry into the joint space.

Many prefer the lateral approach because the needle can “follow” the femoral neck into the joint. The patient is supine, and the hips should be internally rotated - the knees apart and toes touching. A mark is made just anterior to the greater trochanter, and the needle is inserted and directed medially and sightly cephalad toward a point slightly below the middle of the inguinal ligament. One may feel the tip of the needle slide into the joint.

Temporomandibular Joint. For injections, the tempormandibular joint is palpated as a depression just below the zygomatic arch and 1 to 2 cm anterior to the tragus. The depression is more easily palpated by having the patient open and close the mouth. A mark is made and, with the patient's mouth open, a 22-gauge, ½ to 1-inch needle is inserted perpendicular to the skin and directed slightly posteriorly and superiorly.

3. Screening for Compounds that Lubricate the Synovial Tissue

The present invention may be used as an experimental control in assays used for the screening of compounds that may act as therapeutics in the treatment of osteoarthritis. In this regard, the present invention may be used as a known standard in in vitro and in vivo assays known to those practiced in the art.

4. Detecting CACP Protein

The present invention contemplates detecting CACP protein. For example, the present invention contemplates obtaining CACP protein from patients (e.g. from joint tissue or fluid) in order to detect and/or measure CACP protein. Antibodies to CACP can be conveniently used to monitor CACP ad CACP levels. Such assays can be done in liquid or solid phase. For example, the present invention contemplates the use of antibodies to CACP in an ELISA (or similar) format. Alternatively, antibodies to CACP can be used in Western blot assays.

In one embodiment, CACP protein levels are measured to follow the progression of disease. In another embodiment, CACP protein levels are measured to detect the response to treatment.

### EXPERIMENTAL

Materials and Methods

Clinical material

We obtained informed consent from all study participants. Patients were clinically diagnosed as having CACP using published criteria (Bahabri, S. A., et al. “The camptodactyly-arthropathy-coxa vara-pericarditis syndrome. Clinical features and genetic mapping to human chromosome l” Arthritis Rheum. 41:730-735, 1998). The kindred used to reduce the CACP interval to less than 2 Mb has been described previously (family 4; Levick, J. R. Blood flow and mass transport in synovial joints, In Handbook of Physiology Vol. IV, Microcirculation, Part 2. Edited by E. M. Renkins, C. C. Michel, Bethesda, Md., Am. Physiological Society pp 917-947, 1984), as have the clinical descriptions of the two kindreds segregating the 7-bp deletion. The family with the 5-bp deletion is of Brazilian origin. Two sisters, ages 7 and 9, are affected with the disorder. Both had congenital camptodactyly and developed large joint arthropathy in early childhood. Their parents are related as first cousins. The patient with the 41 bp intronic insertion is 22 years old and of American ancestry; he was noted to have bilateral camptodactyly of his thumbs when 6 months old. He developed chronic, painless effusions of both knees and progressive coxa vara deformity as a young child. His parents were consanguineous, but their precise degree of relationship is unknown. The patient with only a single identified heterozygous nonsense mutation is 8 years old and of American ancestry. She has a similarly affected younger male sibling (DNA unavailable for study). Both had congenital camptodactyly and childhood-onset arthropathy. The patient underwent pericardectomy for constrictive pericarditis when age 8 years. Human control synoviocytes were obtained from a 69 year old female patient who underwent total knee arthroplasty for idiopathic osteoarthritis. Bovine tissue was recovered flesh as discarded tissue at the time of necropsy.

Histology

Patient-derived synovium and pericardium was recovered following diagnostic synovial biopsy and therapeutic pericardectomy, respectively; material was fixed in formalin and embedded in paraffin. Cross-sections were stained with hemotoxylin and eosin.

DNA and RNA isolation

Lymphocytes isolated from whole blood were EBV-transformed as previously described (Neitzel H. “A routine method for the establishment of permanent growing lymphoblastoid cell lines” Hum Genet 73:320-326, 1986). and cultured in RPMI containing 10% fetal bovine serum. Human synoviocytes were isolated following a brief incubation of synovial tissue with collagenase (Sigma). Synoviocytes were cultured in DMEM containing 10% fetal bovine serum. DNA was extracted with the Puregene kit (Puregene) and human and bovine RNA were prepared using guanidine-HCl and a CsCl step gradient. We made cDNA with the superscript pre-amplification system (GibcoBRL).

Reduction of the CACP candidate interval

The centromeric end of CEPH mega-YAC 956B9 was cloned using inverse PCR. This YAC contains 3 completely linked simple sequence repeat polymorphisms (D1S191, D1S2848, D1S444) and could contain the centromeric boundary of the CACP interval (see family 4 from: Bahabri, S. A., et al. “The camptodactyly-arthropathy-coxa vara-pericarditis syndrome. Clinical features and genetic mapping to human chromosome 1” Arthritis Rheum. 41:730-735, 1998). Using the end-clone sequence, we designed a PCR primer pair to amplify a 113 bp fragment from genomic DNA in family 4, which is consanguineous. Heterozygosity for SSCP alleles in the affected patient and his mother, indicated that the centromeric end of YAC956B9 lies outside of the CACP minimum interval, which is homozygous by-descent in the patient.

BAC DNA isolation

We used a 40 ml culture of BAC b174L6 to isolate DNA for shotgun library construction using alkaline lysis with an AutoGen 850 automated DNA isolation system following the manufacturer's recommendation (Autogen; Framingham, Mass.). Subsequently the BAC DNA was resuspended in 600 ml dH₂O, treated with RNase (Ambion) and purified over a Microcon 100 column (Amicon)

Shotgun library construction and single stranded DNA isolation

Purified BAC DNA was sent to SeqWright Corporation (Houston, Tex.) for shotgun library construction in M13 phage vector. Approximately 1400 individual M13 plaques were gridded into 96 well microtitre dishes and inoculated with E. coli strain JM101 in 2 X YT media for single-stranded DNA isolation and library storage. We isolated single-stranded DNA in a 96-well format using the High-through Preparation of M13 DNA (THERMOMAX Prep) Protocol from the Washington University Sequencing Center (St. Louis, Mo.).

Sample Sequencing

Single stranded DNA was sequenced using the Energy Transfer fluorescently labeled M13 Forward sequencing primer (Amersham Pharmacia). Briefly, 100 ng of single-stranded template DNA was used in an 8 ml reaction for A/C and 200 ng in 16 ml for G/T with Thermo Sequenase (Amersham Pharmacia). Sequencing reactions were carried out on an ABI CATALYST 800 Molecular Biology LabStation (Perkin Elmer) using the following protocol (95° C. for 5 s, 55° C. for 10 s, 72° C. for 60 s for a total of 15 cycles). The four dye primer reactions were subsequently pooled and precipitated with 132 ml 95% Ethanol and 5 ml Glycogen (Boehringer Mannheim), dried by vacuum and resuspended in 3 ml of loading buffer. Sequencing reactions were electrophoresed in an ABI 377 XL Automated DNA Sequencer (PE Applied Biosystems). We tracked and analyzed the data with DNA Analysis Sequencing Software 3.2 (PE Applied Biosystems).

Mutation detection

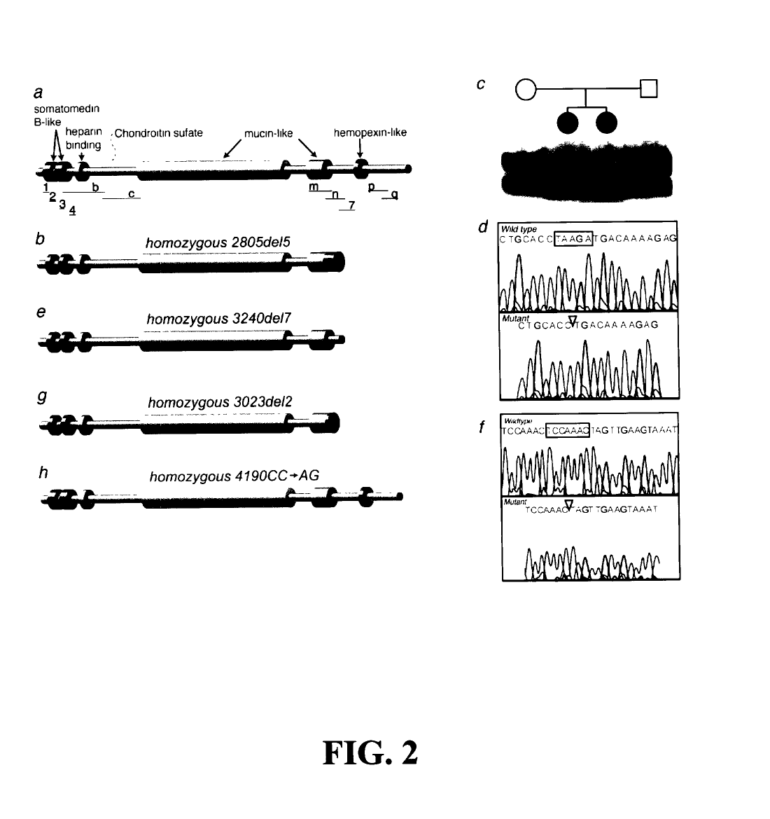

The primers utilized for amplifying CACP from genomic DNA or lymphoblast-derived CDNA are listed in Table 1. FIG. 2a indicates the sites of the amplimers relative to the polypeptide product. Cycling conditions consisted of a 4 min 95° C. initial denaturation, followed by 35 cycles of 95° C. for 30 s, annealing temperature (as indicated in Table 1) for 40 s, 72° C. for 1 min, and a final extension at 72° C. for 10 min. We purified PCR products using Microcon-50 centrifugal filters (Millipore) and sequenced them either with 33P end-labeled primers using the fmol DNA Sequencing System (Promega) or with an ABI 377 with labeled di-deoxy terminators. Fifty unaffected and unrelated control DNA samples were also screened for mutations.

Sequence analysis

Data generated through systematic BAC clone sequencing was analyzed using WebBLAST (Ferlanti, E. S., et al., “WebBLAST 2.0: An Integrated Solution for Organizing and Analyzing Sequence Data” Bioinformatics 5:422-423, 1999). Upon generation of BAC clones giving sufficient coverage, data was exported from WebBLAST and assembled using the PHRED/PHRAP/CONSED suite (Ewing, B., et al. “Base-calling of automated sequencer traces using PHRED” Genome Res. 8:175-85, 1998; Gordon, D., et al. “CONSED: A graphical tool for sequence finishing” Genome Res. 8:195-202, 1998).

Northern blot analysis

We probed a bovine northern blot and a human multiple-tissue northern blot (Clontech) with a 681-bp DNA fragment generated from human synoviocyte cDNA using MFOR and NREV as primers (see Table 1). The probe was purified using a Microcon-50 Centrifugal Filter Device (Millipore) and then 32P dCTP labeled by random priming with the High Prime (Boehringer Mannheim). Hybridization was performed at 68° C. in ExpressHyb buffer (Clontech) and washed at a final stringency of 0.1X SSC at 50° C. for 40 minutes. Blots were exposed to a phosphor screen (Molecular Dynamics) and then quantified by using the manufacturer's ImageQuant software. A control actin probe was also tested, following the manufacturer's recommended protocol.

### Example 1

The CACP locus has been mapped to a 1.9 cM genetic interval on human chromosome 1q25-q31 (Levick, J. R. Blood flow and mass transport in synovial joints, In Handbook of Physiology Vol. IV, Microcirculation, Part 2. Edited by E. M. Renkins, C. C. Michel, Bethesda, Md., Am. Physiological Society pp 917-947, 1984). Using an informative simple sequence repeat polymorphism derived from an end-clone of CEPH mega-YAC 956-B9, the CACP candidate interval could be reduced to less than 2 Mb (data not shown). We constructed a complete BAC contig across the critical region and performed sample sequencing to identify novel polymorphic markers, as well as candidate genes within this interval. The assembled genomic sample sequence of the human BAC clone b174L6 was BLAST searched to find homologous sequences in the public databases using WebBLAST (Ferlanti, E. S., et al, “WebBLAST 2.0: An Integrated Solution for Organizing and Analyzing Sequence Data” Bioinformatics 5:422-423, 1999; http://genome.nhgri.nih.gov/webblast/). BLASTN identified a human EST (AA377436) derived from synovial tissue cDNA having 100% identity to our query sequence. This EST is 98% identical to the human megakaryocyte growth and stimulating factor precursor (MSF). The full length cDNA coding sequence that contains this EST is virtually identical to that of MSF (Genbank accession number U70136), leading us to conclude that CACP and MSF are the same. A putative bovine ortholog of this gene has been called “superficial zone protein” (SZP) (Flannery, C. R. et al. “Articular cartilage superficial zone protein (SZP) is homologous to megakaryocyte stimulating factor precursor and is a multifunctional proteoglycan with potential growth-promoting, cytoprotective, and lubricating properties in cartilage metabolism” Biochem. Biophys. Res. Commun. 254:535-541, 1999; Schumacher B. L., et al. “A novel proteoglycan synthesized and secreted by chondrocytes of the superficial zone of articular cartilage” Arch. Biochem. Biophys. 311:144-152, 1994). This protein is synthesized by chondrocytes in the superficial zone of articular cartilage (closest to the joint cavity) and by joint synoviocytes (Schumacher, B. L., et al. “Immunodetection and partial cDNA sequence of the proteoglycan, superficial zone protein, synthesized by cells lining synovial joints” J. Orthop. Res. 17:110-120, 1999). Until now, the function of the CACP gene product has not been elucidated. In the examples of the present invention, we show that the CACP protein functions as a joint “lubricant” and defective CACP is the causative factor in CACP.

### Example 2

We used PCR and RT-PCR to amplify portions of CACP from patient-derived genomic DNA and mRNA, respectively, and identified four likely disease-causing mutations (FIG. 2); none of these mutations was observed in 100 control chromosomes. A homozygous 5-bp deletion (FIG. 2d) is present in two siblings whose parents are consanguineous. This mutation creates a frame-shift that truncates the polypeptide chain at 974 amino acid residues after altering the carboxyl 39 residues (FIG. 2b). The mutant allele co-segregates with the phenotype (FIG. 2c). In two consanguineous kindreds that share a common disease-associated haplotype [families 2 and 3 (Bahabri, S. A., et al. “The camptodactyly-arthropathy-coxa vara-pericarditis syndrome. Clinical features and genetic mapping to human chromosome 1” Arthritis Rheum. 41:730-735, 1998)], affected individuals have a homozygous 7-bp deletion (FIG. 2f) creating a frameshift that truncates the protein by 320 amino acid residues (FIG. 2e). The mutation co-segregates with the phenotype in both families (data not shown). The third mutation, also found in a patient whose parents are consanguineous, is a homozygous 41-bp intronic insertion; occurring 14 residues upstream of a 3′ splice-acceptor site, the insertion disrupts the splice site's polypyrimidine tract (FIG. 2g). A fourth mutation was observed in a patient whose parents are non-consanguineous. This individual inherited a C to T transition at nucleotide 724 from one unaffected parent. The mutation creates a nonsense codon (TAA) and is predicted to terminate protein translation after only 241 amino acid residues (FIG. 2h). We have not yet found a disease causing mutation in this patient's other allele, nor in four other CACP kindreds [families 1 and 4 (Bahabri, S. A., et al. “The camptodactyly-arthropathy-coxa vara-pericarditis syndrome. Clinical features and genetic mapping to human chromosome 1” Arthritis Rheum. 41:730-735, 1998) and two other unpublished cases]. The likely reason for this is that CACP cDNA contains a 2.0 kb region encoding the protein's highly repetitive mucin-like domain, which has proven difficult to PCR amplify and sequence (FIG. 2a). Finding four different CACP mutations in patients with CACP strongly supports the gene's causative role in the pathogenesis of the disorder. Thus far, all identified mutations are predicted to cause truncations in the protein (FIG. 2). The mechanism by which mutations in CACP cause the CACP phenotype is unknown. However, the absence of heterozygote manifestations suggests the mutations cause a loss of protein function, rather than a gain of new function.

### Example 3

An amino-terminal portion of the CACP protein was initially purified as a ˜30 kDa polypeptide capable of stimulating megakaryocyte growth (Turner, K. J., et al. “Purification, biochemical characterization, and cloning of a novel megakaryocyte stimulating factor that has megakaryocyte colony stimulating activity” Blood 78(suppl. 1): pp. 279, 1991). Subsequently, the fragment was found to derive from a highly glycosylated precursor protein with an apparent molecular mass of ˜400 kDa (Merberg, D. M. et al., In Biology of Vitronectins and Their Receptors, Edited by K. T. Preissner, S. Rosenblatt, C. Kost, J. Wegerhoff & D. F. Mosher Elsevier Science, B.V., pp 45-52, 1993). The tissue origin of the precursor protein and the means by which its biologically active fragment is derived has not been reported. This protein was also identified as a ˜345 kDa proteoglycan synthesized by chondrocytes residing in the superficial zone of bovine articular cartilage and by some intimal synoviocytes (Schumacher B. L., et al. “A novel proteoglycan synthesized and secreted by chondrocytes of the superficial zone of articular cartilage” Arch. Biochem. Biophys. 311:144-152, 1994; Schumacher, B. L., et al. “Immunodetection and partial cDNA sequence of the proteoglycan, superficial zone protein, synthesized by cells lining synovial joints” J. Orthop. Res. 17:110-120, 1999). The proteoglycan is substituted with both chondroitin sulfate and keratan sulfate and is heavily modified with O-linked oligosaccharides in mucin-like repeat domains (Flannery, C. R. et al. “Articular cartilage superficial zone protein (SZP) is homologous to megakaryocyte stimulating factor precursor and is a multifunctional proteoglycan with potential growth-promoting, cytoprotective, and lubricating properties in cartilage metabolism” Biochem. Biophys. Res. Commun. 254:535-541, 1999). DNA and protein sequence homologies indicate that bovine superficial zone protein is orthologous to CACP (megakaryocyte stimulating factor precursor) (Schumacher B. L., et al. “A novel proteoglycan synthesized and secreted by chondrocytes of the superficial zone of articular cartilage” Arch. Biochem. Biophys. 311:144-152, 1994).

### Example 4

The identification of CACP mutations should help delineate the protein's normal function. CACP appears to encode a novel type of proteoglycan. Its predicted peptide sequence does not contain membrane-spanning domains found in cell surface receptor proteoglycans, such as syndecans, CD44, and NG2 (Woods A., and Couchman J. R. “Syndecans: synergistic activators of cell adhesion” Trends Cell Biol. 8:189-92, 1998; Ponta, H., et al. “The CD44 protein family” Int. J. Biochem. Cell Biol. 30:299-305, 1998; Nishiyama, A. et al. “The primary structure of NG2, a novel membrane-spanning proteoglycan” J. Cell Biol. 114:359-371, 1991), nor does it appear to be covalently linked to membranes like the glypicans (David, G. “Biology and pathology of the pericellular heparan sulphate proteoglycans” Biochem. Soc. Trans. 19:816-820, 1991). Its secretion into the joint cavity distinguishes it from cartilage matrix bound proteoglycans such as aggrecan and the small leucine-rich proteoglycans decorin, fibromodulin and lumican, which are primarily retained in the cartilage matrix through interactions with hyaluronan and fibrillar collagens, respectively (Iozzo R. V. “Matrix proteoglycans: from molecular design to cellular function” Ann. Rev. Biochem. 67:609-52, 1998). Due to its high glycosylation content and mucin-like repeats, CACP-1 may act as a joint/intimal cell lubricant. Both synovial and pericardial cell hyperplasia could represent secondary consequences of insufficient cell surface lubrication. The slowly progressive nature of the arthropathy in patients affected with CACP and the incomplete penetrance for symptomatic pericardial involvement would support this hypothesis. However, cell overgrowth may be primary to the pathogenesis of the disorder. Two unrelated patients in our series had multiple small ganglion cysts (lesions adjacent to tendon sheaths filled with mucinous material) which may result from dysregulated synovial cell growth. Also, supporting a regulatory role for the CACP protein product is the occurrence of coxa vara deformity (angular deformation of the hips) (Bulutlar, G., et al., “A familial syndrome of pericarditis, arthritis, camptodactyly, and coxa vara” Arthritis Rheum. 29:436-438, 1986), a primary developmental defect of the femoral neck, and peri-articular osteoporosis.

### Example 5

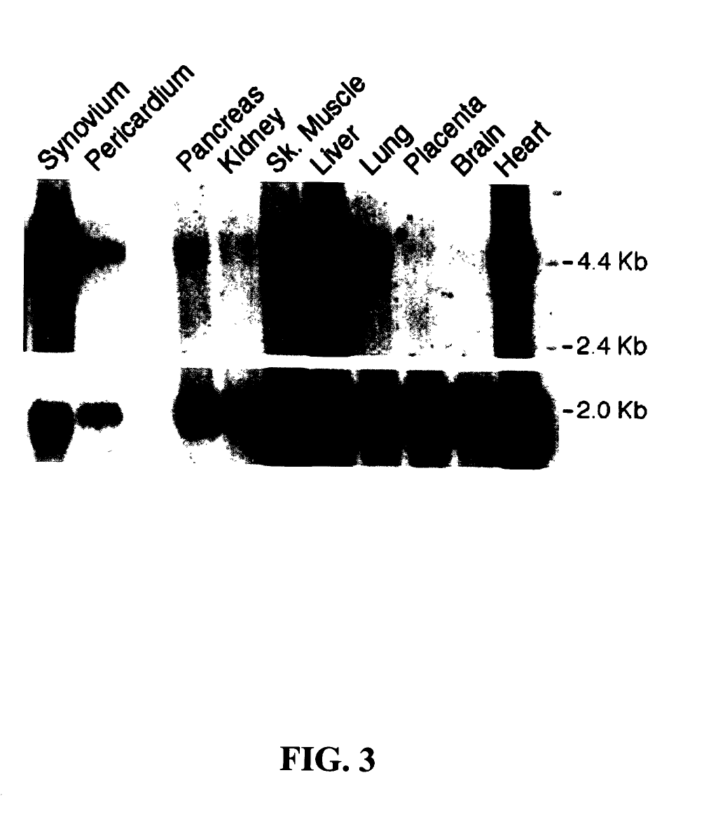

One important role for CACP may involve the regulation of intimal cell growth. Synoviocyte hyperplasia and, less commonly, hyperplasia of other intimal cell layers (pericardium and pleura) occurs in rheumatoid arthritis (RA), suggesting that a disease associated disruption of CACP's regulatory function could also contribute to the pathogenesis of RA. It is interesting that the 30 kDa megakaryocyte stimulating factor fragment was found in serum and in urine (Merberg, D. M. et al., In Biology of Vitronectins and Their Receptors Edited by K. T. Preissner, S. Rosenblatt, C. Kost, J. Wegerhoff & D. F. Mosher Elsevier Science, B.V., pp 45-52, 1993). CACP is abundantly expressed in synovial tissue (FIG. 3A). Additionally, on a commercially available multi-tissue northern blot CACP mRNA is observed in other several other tissues, including liver (FIG. 3B).

It should be clear from the above that the present invention provides reagents and methods for the screening of compounds that can be used as therapeutics for Osteorthritis, as well as providing reagents and methods for the treatment of Osteorthritis.

TABLE 1

CACP Primers

| | | Forward, | Anneal | Size³ |
|---|---|---|---|---|
| Exon¹ | Location² | Reverse | (° C.) | (bp) |
| 1 | −73 → −54 | 1FOR 5′-gcaatcctaagttaatggtg-3′ | 50 | (227) |
| | +60 → +41 | 1REV 5′-atcaagactgaatgattagc-3′ | | |
| 2 | −47 → −28 | 2FOR 5′-ctataaagtggtttggccat-3′ | 55 | (218) |
| | +48 → +31 | 2REV 5′-gactcgcagtgttgcttg-3′ | | |
| 3 | −48 → −29 | 3FOR 5′-ctggcttcacaatgaataat-3′ | 55 | (230) |
| | +61 → +43 | 3REV 5′-ctaaggtaggtgcacagat-3′ | | |
| 4 | −30 → −31 | 4FOR 5′-gccgatgaacataaacaaga-3′ | 55 | (286) |
| | +86 → +67 | 4REV 5′-tctctgagaatgggcttaga-3′ | | |
| 7 | −84 → −65 | 7FOR 5′-gaaccatgtggaaagacttg-3′ | 55 | (226) |
| | +64 → +45 | 7REV 5′-ctttggttctcataaatgcc-3′ | | |
| 4*/5/ | 330 → 347 | BFOR 5′-cacatcaccaccatcttc-3′ | 55 | 389 |
| 6* | 718 → 699 | BREV 5′-tagacgtgtcaggagttgtg-3′ | | (>1.5kb) |
| 6* | 655 → 674 | CFOR 5′-gtagatgaagctggaagtgg-3′ | 55 | 354 |
| | 1008 → 991 | CREV 5′-ttcagctttgggtgtagg-3′ | | |
| 6* | 2787 → 2804 | MFOR 5′-aactacaactgctgcacc-3′ | 55 | 351 |
| | 3137 → 3120 | MREV 5′-ggttttctcactctaggc-3′ | | |
| 6*/7* | 3065 → 3084 | NFOR 5′-aaaagccaaccaaagcaccc-3′ | 60 | 403 |
| | 3467 → 3448 | NREV 5′-gtagtcagtccatctactgg-3′ | | (˜1kb) |
| 9*/10/ | 3707 → 3724 | PFOR 5′-ttggaggactaactggac-3′ | 55 | 489 |
| 11/12* | 4195 → 4178 | PREV 5′-ctttggataaggtctgcc-3′ | | (>1.5kb) |
| 11*/12 | 4077 → 4096 | QFOR 5′-cagaaaacctgacggctatg-3′ | 58 | 394 |
| | 4470 → 4451 | QREV 5′-tttacaggtgtgagccatgc-3′ | | (˜1kb) |

¹Asterisks (*) indicate that only portions of these exons were amplified from the cDNA with the primer pair.
²The location of the forward intronic primer is relative to either to the exon's 5′ boundary, or in exon 1, the translation initiation codon. The location of the reverse intronic primer is relative to the exon's 3′ boundary. The location of exonic primer is relative to the adenosine residue within methionine translation initiation codon.
³Amplimer sizes within parentheses correspond to products derived from genomic DNA template. Amplimer sizes that are not in parentheses are derived from cDNA as template.

1. A method of treating a subject, comprising:
a) providing: i) a subject, and ii) a preparation comprising the CACP protein, or portion thereof; and
b) administering said preparation to said subject.

2. The method of claim 1, wherein said administering comprises intra-articular injection.

3. The method of claim 1, wherein said administering comprises intravenous injection.

4. The method of claim 1, wherein said preparation further comprises a local anesthetic.

5. A method of treating a subject, comprising:
a) providing: i) a subject with symptoms of osteoarthritis, and ii) a preparation comprising the CACP protein, or portion thereof; and
b) administering said preparation to said subject under conditions such that said symptoms are reduced.

6. The method of claim 5, wherein said administering comprises intra-articular injection.

7. The method of claim 5, wherein said administering comprises intravenous injection.

8. The method of claim 5, wherein said preparation further comprises a local anesthetic.

9. A composition, comprising CACP protein, or portion thereof, in combination with an anesthetic.
